(19) United States
(12) Patent Application Publication
(10) Pub. No.: US 2025/0127200 A1
(43) Pub. Date: Apr. 24, 2025

(54) PLANT-BASED WALL MATERIALS AND EMULSIFIERS AND THE USES AND METHODS OF MANUFACTURE THEREOF

(71) Applicant: Purdue Research Foundation, West Lafayette, IN (US)

(72) Inventor: Yuan YAO, West Lafayette, IN (US)

(21) Appl. No.: 18/686,410

(22) PCT Filed: Aug. 25, 2022
(86) PCT No.: PCT/US2022/041507

§ 371 Date: Feb. 24, 2024

Related U.S. Application Data
(60) Provisional application No. 63/236,785, filed on Aug. 25, 2021.

(51) Int. Cl. A23L 29/10; A23L 7/10; A23L 27/00

(52) U.S. Cl. CPC A23L 29/10; A23L 7/10; A23L 27/72

(57) ABSTRACT

Wall materials and emulsifiers that comprise a material separated or isolated from a specific genotype of cereals, compositions, products, and encapsulation and/or emulsification methods.

### PRIORITY

This patent application is related to and claims the priority benefit of U.S. Provisional Patent Application No. 63/236,785 filed Aug. 25, 2021. The content of the foregoing application is hereby incorporated by reference in its entirety into this disclosure.

### TECHNICAL FIELD

The present disclosure generally relates to natural, plant-based emulsifiers and/or encapsulation wall materials, emulsions, encapsulated compositions, products comprising the wall materials, emulsifiers, and/or the compositions of encapsulation systems, and methods for manufacturing the same. More particularly, the plant-based materials and compositions comprise a portion or an isolate of kernels of sugary-1 (su1) cereal, such as su1 maize (corn). These materials and compositions can be used for emulsification, emulsion stabilization, encapsulation, or microencapsulation, and/or a combination thereof, and are highly functional and sustainable. The su1 cereal-formulated products can be used in the areas of food and beverage, cosmetics, personal care, home care, agriculture, and pharmaceuticals.

### BACKGROUND

This section introduces aspects that may help facilitate a better understanding of the disclosure. Accordingly, these statements are to be read in this light and are not to be understood as admissions about what is or is not prior art.

Emulsion-forming and stabilization and spray-dry (micro)encapsulation (SD-M) are multi-billion-dollar industries that enable or support formulations for active ingredients, nutrients, flavors, colorants, oils, and other components used broadly for food and beverages, cosmetics, personal care, home care, agricultural or crop care, and pharmaceutical applications.

In SD-M products, the core materials (i.e., the ingredients or components to be encapsulated and/or protected) can be embedded in wall materials for prolonged protection and convenient use. As major ingredients for processed food, SD-M wall materials face strong industrial and consumer demands for being clean-label, plant-based, and sustainable, while still providing superior functionalities.

SD-M has been used to formulate both hydrophilic (i.e., water-soluble) and lipophilic (i.e., oil- or lipid-soluble) ingredients. For SD-M of hydrophilic ingredients, wall materials are desired to be soluble or dispersible with low viscosity, hygroscopicity, and oxygen permeation. For SD-M of lipophilic ingredients, the technical requirements of the formulation system are more complicated in that an emulsifier or emulsifiers are needed for emulsification and emulsion stabilization before spray-drying. Indeed, such mixes generally include emulsifiers as essential ingredients. The emulsifiers are added to control or impart desirable end-product attributes such as volume, moistness, tenderness, evenness, smoothness, high stability, and other qualities. It is highly desirable that a wall material is also an effective emulsifier such that it can be used with both lipophilic and hydrophilic core materials.

There are several conventionally available wall materials in the food industry. Maltodextrin, inulin, and pea protein, for example, are wall materials that are commonly used, but that have no or low emulsification properties. Accordingly, when used as wall materials, they additionally require an emulsifier for SD-M of lipophilic ingredients. Acacia gum (gum arabic), octenyl succinic anhydride (OSA) modified starch (OSA-starch), sodium caseinate, and whey protein are wall materials with emulsification properties, but with different limitations. In general, none of the wall materials commercially available are natural, plant-based, functional, and sustainable. Acacia gum is naturally occurring, but its availability, affordability, and sustainability are in question. OSA-starch, a chemically modified starch, is a billion-dollar-market ingredient (see Marz, Starches/Glucose: Global Markets, BCC Research Market Forecasting, p. 78 (2013)). However, OSA-starch does not have a clean label and has undergone increasing scrutiny by end-users. Pea protein is plant-based, but with limitations on viscosity, solubility, and emulsification capability. In the industry as a whole, there are unmet needs for natural, plant-based, functional, and sustainable wall materials. What is needed is a plant-based, clean, functional, cost-effective and sustainable wall material for encapsulating lipophilic, hydrophilic, and other types of core materials.

### SUMMARY

Wall materials and emulsifiers are provided. In certain embodiments, the wall material or emulsifier comprises a portion or an isolate from sugary-1 (su1) cereal, wherein the portion or isolate comprises greater than about 10% phytoglycogen, greater than about 0.1% soluble proteins, and less than about 60% saccharides with a degree of polymerization (DP) equal to or lower than 2. In certain embodiments, the portion or isolate comprises greater than about 20% phytoglycogen, greater than about 0.5% soluble proteins, and less than about 50% saccharides with a DP equal to or lower than 2. The portion or isolate can further comprise less than about 80% starch. In certain embodiments, the portion or isolate further comprises about 0-80% of starch. In certain embodiments, the portion or isolate further comprises less than about 40% of starch.

The cereal can be corn, rice, barley, or sorghum, for example. In certain embodiments, the cereal is not subjected to germination treatment. In certain embodiments, the cereal is subjected to germination treatment (e.g., prior to processing). In certain embodiments, a portion or an isolate from sugary-1 cereal comprises endosperm fractions.

In certain embodiments, a 30% dispersion (by weight) of said wall material or emulsifier in water has a viscosity not greater than about 500 mPas.

The wall material or emulsifier can further comprise one or more core material encapsulated in the wall material or emulsifier. In certain embodiments, the one or more core material comprises one or more lipophilic components. In certain embodiments, the one or more core material comprises one or more hydrophilic components. The one or more core material can be one or more of a food ingredient, a pharmaceutical compound, an oil, a nutrient, a fine chemical, a preservative, a colorant, an enzyme, a microorganism, a flavor, and/or an active ingredient.

The wall material or emulsifier hereof can be used for emulsification, emulsion stabilization, encapsulation, and/or microencapsulation. The wall material or emulsifier hereof can be for use in a food and beverage, cosmetics, personal care, home care, agricultural or crop care, or pharmaceutical applications. The wall material or emulsifier hereof can be for use in a food and beverage, cosmetics, personal care, home care, agricultural or crop care, or pharmaceutical applications for the emulsification, emulsion stabilization, encapsulation, and/or microencapsulation of one or more nutrients, one or more ingredients, one or more lipophilic or hydrophilic components, and one or more fine chemicals. The wall material or emulsifier hereof can be for emulsification, emulsion stabilization, encapsulation, and/or microencapsulation of one or more flavors, colorants, enzymes, microorganisms, oils, preservatives, active ingredients, or other ingredients. The wall material or emulsifier hereof can be used for emulsification, emulsion stabilization, encapsulation, and/or microencapsulation of one or more food components, nutrients, cosmetic or personal care components, colorants, oleoresins, essential oils, enzymes, microorganisms, active ingredients, drugs, home care chemicals, agricultural or crop care chemicals, fine chemicals, or a combination thereof. The wall material or emulsifier hereof can be for use in a spray-dry operation.

Compositions are also provided. In certain embodiments, the composition comprises a wall material or emulsifier separated or isolated from su1 cereal, and one or more core materials mixed with, emulsified by, coated by, or entrapped within the wall material or emulsifier. The cereal of the wall material or emulsifier can be, for example, corn, barley, sorghum, or rice. The cereal can be subjected to germination treatment. In certain embodiments, the cereal is not subjected to germination treatment.

The wall material or emulsifier can comprise greater than about 10% phytoglycogen, greater than about 0.1% soluble proteins, and less than about 60% saccharides with a degree of polymerization (DP) equal to or lower than 2. The wall material or emulsifier can comprise greater than about 20% phytoglycogen, greater than about 0.5% soluble proteins, and less than about 50% saccharides with a DP equal to or lower than 2.

The one or more core material of the composition can comprise one or more food or beverage, cosmetic, personal care, home care, agricultural or crop care, or pharmaceutical components, one or more nutrients, one or more colorants, one or more flavors, one or more oils, one or more enzymes, one or more microorganisms, one or more active ingredients, one or more fine chemicals, or a combination of any of the foregoing.

The wall material or emulsifier can comprise less than about 80% of starch (by weight).

The composition can be (e.g., formulated as), for example, a liquid, a gel, a syrup, a paste, a solid, a powder, or an inhalant.

In certain embodiments, the composition is spray-dried.

Methods of encapsulating one or more core materials are provided. In certain embodiments, the method of encapsulating one or more core materials comprises: providing a liquid feed comprising at least one core material dispersed in a solution, the solution comprising a solvent and a wall material or emulsifier; and spray-drying the liquid feed to form a solid product. The wall material or emulsifier can comprise a portion or an isolate from su1 cereal; and greater than about 10% phytoglycogen, greater than about 0.1% soluble proteins, and less than about 60% saccharides with a DP equal to or lower than 2.

The liquid feed can be an aqueous emulsion and at least one of at least one core material is lipophilic. In certain embodiments, at least one core material is hydrophilic. In certain embodiments, at least one core material is lipophilic.

A portion or an isolate from su1 cereal can comprise endosperm fractions. The cereal can be, for example, corn, barley, sorghum, or rice.

In certain embodiments, the solid product is in a powder or granulated form.

In certain embodiments, the solvent of the liquid feed comprises water.

Food products, nutraceuticals, and dietary products are also provided. In certain embodiments, the food product, nutraceutical, or dietary product comprises the wall materials or emulsifiers and/or the compositions described herein. Cosmetic, personal care, and home care products comprising the wall materials or emulsifiers and/or the compositions are also provided.

Pharmaceutical products comprising the wall material or emulsifier and/or compositions are further provided. Still further, agricultural or crop care products comprising the wall material or emulsifier and/or compositions are provided.

Methods for preparing an emulsion are also provided. In certain embodiments, such methods comprise providing a mixture of at least one core material dispersed in a solution, wherein the solution comprises a solvent and a wall material or emulsifier. The wall material or emulsifier can be any of the wall materials or emulsifiers described herein. In certain embodiments, the wall material or emulsifier comprises a portion or an isolate from su1 cereal; and greater than about 10% phytoglycogen, greater than about 0.1% soluble proteins, and less than about 60% saccharides with a degree of DP equal to or lower than 2.

The cereal can be corn, rice, barley, or sorghum. The su1 cereal can be subjected to germination treatment. In certain embodiments, the su1 cereal is not subjected to germination treatment. A portion or isolate from the su1 cereal can comprise endosperm fractions.

In certain embodiments, the portion or isolate can comprise greater than about 20% phytoglycogen, greater than about 0.5% soluble proteins, and less than about 50% saccharides with a DP equal to or lower than 2. In certain embodiments, the portion or isolate further comprises less than about 80% starch, and optionally less than about 40% of starch. In certain embodiments, the portion or isolate further comprises about 0-80% of starch.

In certain embodiments, at least one of the core materials is lipophilic.

### BRIEF DESCRIPTION OF DRAWINGS

FIG. 1 shows images of the fraction of su1 corn kernels.

FIG. 2 shows a spray-drying system (left) and structures of spray-dry (micro)encapsulation (SD-M) particulate of hydrophilic (water-soluble) and lipophilic ingredients (right).

FIG. 3 shows images of blueberry fluid (top) and beet juice (bottom) SD-M solids (su1 corn extract as wall material) and the materials in 5% dispersions (in the vials).

FIG. 4 shows an image of freshly prepared emulsion of paprika oleoresin using su1 corn extract-1, su1 corn extract-2, su1 corn extract-3, acacia gum, and Hi-Cap 100.

FIG. 5 shows images of the diluted paprika oleoresin emulsions formed by Hi-Cap 100 and su1 corn extract-1, su1 corn extract-2, and su1 corn extract-3, with the top row showing diluted emulsions photographed right after dilution and the bottom row showing diluted emulsions photographed five days after dilution. The light intensity was about 310 lux, and the initial content of paprika oleoresin was the same for each sample.

FIG. 6 shows the impact of heat treatment on the color of paprika oleoresin SD-M solids prepared using su1 corn extract-2 (E2) or Hi-Cap 100 (HC). The wall material-to-oil ratio was 2:1.

For the heat treatment, original (top) spray-dried solids (tested in duplicates) were placed at 70° C. for 8 hours and then allowed to cool to room temperature in 16 hours (bottom). A portion of E2 and HC images were magnified for both original and heated groups (right).

FIG. 7 depicts the emulsion feeds, SD-M solids, and rehydrated dispersions (emulsions) of orange oil and lutein oil formed using su1 corn extract. The oil/wall-material/water ratio was 20/20/100 for orange oil and 9.6/26.7/100 for lutein oil. Each rehydrated dispersion contained 5% SD-M by weight.

### DETAILED DESCRIPTION

While the concepts of the present disclosure are illustrated and described in detail in the description herein, results in the description are to be considered as exemplary and not restrictive in character; it being understood that only the illustrative embodiments are shown and described, and that all changes and modifications that come within the spirit of the disclosure are desired to be protected.

In the food and beverage, cosmetics, personal care, home care, agricultural or crop care, pharmaceutical, and other industries, emulsifications and/or (micro)encapsulations (i.e., encapsulation and/or microencapsulation) are broadly used for enabling or supporting various formulations for greater stability, better quality, higher bioavailability, prolonged shelf life, controlled release of the core material, and other desirable attributes. For emulsification and emulsion stabilization, naturally occurring, sustainable, and cost-effective emulsifiers are highly desirable for both food and non-food industries. In the food and non-food industries, the goal of (micro)encapsulation is to form a protecting layer, coating, or matrix for a target component (core material). Such protections are usually used to prevent or reduce quality deterioration of the core material caused by chemical degradation or unwanted release. For example, (micro)encapsulation can provide high-quality food ingredients through better retentions of flavor and taste, higher stabilities of active ingredients, and improved bioavailability of nutrients. In addition, (micro)encapsulation can also be used to immobilize and deliver enzymes or cells such as probiotics. As used herein, “microencapsulation” means when the particle size of an encapsulation solid is at the micron level.

According to BCC Research, the global sales of encapsulated food products were around $20.5 billion in 2011 and expected to reach $36.7 billion in 2017 (Dewan, Global markets for controlled intelligent packaging, preservation and shelf-life extension for food and beverages—focus on encapsulation, irradiation and other applications, BCC Research Market Forecasting (2013)). Among different regions, North America dominated the encapsulated food and beverage market with $8.1 billion in 2011 and predicted $14.4 billion in 2017 (see Dewan (2013), supra, page 140).

### Wall Materials and Emulsifiers

Novel wall materials and emulsifiers (e.g., for use in encapsulation and microencapsulation of core materials) are provided. The wall materials or emulsifiers hereof can be used for emulsification, emulsion stabilization, and encapsulation and/or microencapsulation.

The costs of production of the wall materials or emulsifiers are low, and the manufacturing processes are sustainable. As used herein, “core material” means a material to be dispersed, suspended, dissolved, emulsified, coated, embedded, encapsulated, protected, and/or stabilized in a system that comprises a wall material or emulsifier. The core material can be a solid, semi-solid, gel, paste, syrup, liquid, gas, or other forms, or a combination thereof, and can be hydrophilic (i.e., water-soluble), lipophilic (i.e., lipid- or oil-soluble), solvent-soluble, or not soluble (e.g., crystallites or metal particles that form suspensions, but not solutions or dispersions).

The terms “wall material” and “emulsifier” and their variants mean a material that displays the functional properties described herein and that can be used to encapsulate, coat, embed emulsify, protect, and/or stabilize a core material. As used herein, the terms “wall material” and “emulsifier” are interchangeable, except where expressly stated otherwise.

The wall material or emulsifier comprise a portion or an isolate from the kernels of sugary-1 (sugary1 or su1) cereal, that is, a cereal with its genotype containing the mutation of sugary-1 either alone or with other mutations. Examples of su1 cereal, without limitation, are su1 corn (maize), sorghum, barley, or rice. The su1 cereal can be any su1 cereal that produces phytoglycogen (PG). As used herein, the term “su1 cereal” or variants thereof (e.g., such as a particular kind of cereal) indicate the whole or a portion of the su1 plant or the kernels of su1 cereal. For example, unless otherwise indicated, “su1 com” means both the whole su1 corn plant or kernels of su1 corn. In certain embodiments, the wall material or emulsifier comprises a whole kernel of a su1 cereal.

In certain embodiments, the su1 cereal is su1 corn. In certain embodiments, the su1 cereal is su1 rice. The su1 cereal can be subjected to a germination treatment or not subjected to germination treatment.

In certain embodiments, a portion or an isolate from su1 cereal comprises endosperm fractions. In certain embodiments, a portion or an isolate from su1 cereal comprises not only the fractions from endosperm, but also the fractions from germs and/or bran.

As a traditional (or “normal”) sweet corn, su1 corn (also called SU or su corn) has been a pleasant staple vegetable since the last century. For a su1 cereal, the sweetness results from the sugar accumulation due to a reduced starch biosynthesis resulting from the deficiency of an isoamylase-type starch debranching enzyme, and carbon flux is at least partially directed to the genesis of PG, a dendrimer-like, high-molecular-weight alpha-glucan that is structurally similar to glycogen (phytoglycogen meaning “plant-based glycogen”) (see Dinges et al., Molecular structure of three mutations at the maize sugary1 locus and their allele-specific phenotypic effects, Plant Phys 125: 1406-1418 (2001); and Huang and Yao, Particulate structure of phytoglycogen nanoparticles probed using amyloglucosidase, Carbohydrate Polymers 83, 1665-1671 (2011)).

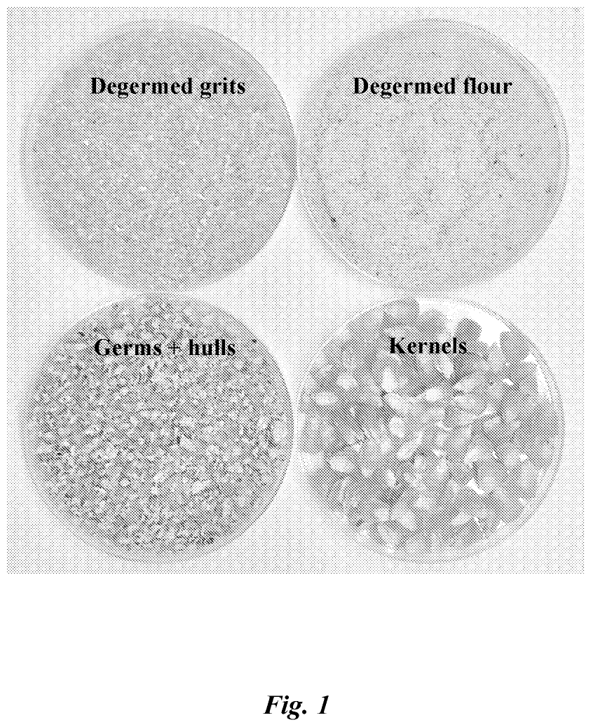

FIG. 1 shows images of kernels and kernel fractions of su1 corn. Degermed fractions include degermed grits and/or flour (named based on the size of the particles), germs, and hulls. The most abundant component in su1 corn endosperm is PG. In mature (dry) su1 corn kernels, PG is about 25-30% of kernel weight, whereas starch is about 10%. Meanwhile, there can be about 5-20% sugars (e.g. saccharides), which can contain disaccharides (degree of polymerization of 2 (DP2)) and monosaccharides (degree of polymerization of 1 (DP1)). The amount of proteins in mature su1 corn kernels ranges from about 12-15% (see Goldman and Tracy, Kernel protein concentration in sugary-1 and shrunken-2 sweet coRN, HortScience 29: 209-210 (1994)), which is higher than about 7-10% for field corns (see Ulger et al., Influence of nitrogen rates and row spacing on corn yield, protein content, and other plant parameters, J of Plant Nutr. 20: 1697-1709 (1997)).

Surprising results of the studies described herein were that a portion or an isolate from the kernels of su1 cereal, such as su1 corn, displayed functional properties of: (1) emulsification; (2) emulsion stabilization; and (3) (micro)encapsulation (i.e., encapsulation and/or microencapsulation). Therefore, a portion or isolate from (e.g., the kernels of) su1 cereal can be used as a novel wall material, a novel emulsifier, or both.

### Preparation Approaches

There are multiple approaches to prepare the wall materials or emulsifiers from a portion or an isolate of su1 cereal (each referred to herein as a “preparation approach”). In certain embodiments, the preparation approach can comprise using flour or grits of whole kernels of su1 cereal, either directly as the wall material or emulsifier or as a starting material for obtaining or extracting the wall material or emulsifier.

In certain embodiments, the preparation approach can comprise isolating a fraction from su1 cereal that contains endosperm components. Such isolation can be achieved using dry methods such as one or more of dry milling, grinding, separation based on density, particle size, or color, and/or using any other dry method now known or hereinafter developed in the relevant arts.

The isolation of a fraction that contains endosperm components can also be achieved using wet methods (i.e., involving the use of water or in another aqueous system). For example, and without limitation, such wet methods can include one or more of wet milling, mixing with water, blending, homogenization, separation based on density (e.g., centrifugation or cyclone separation) and/or particle size (e.g., filtration, microfiltration, or ultrafiltration), extraction, heat treatment, shear treatment, drying (e.g., spray-drying, freeze drying, drum-drying, or vacuum-drying), powder processing, and/or granulation. Methodologies and protocols for these isolation techniques are commonly known in the art and will be readily understood by someone of ordinary skill in view of the present disclosure.

In certain embodiments, the preparation approach can comprise a combination of one or more of the aforementioned approaches (e.g., use of the flour or grits of whole kernels, isolation of a faction using dry methods, and/or isolation of a fraction using wet methods).

Pre-treatments of the su1 cereal resulting in various degrees of germination (each, a “germination treatment”) can also be employed prior to processing using the preparation approach(es). In certain embodiments, no germination treatment is applied (i.e., the su1 cereal kernels are not in contact with water and/or placed in a high-humidity environment prior to processing). Alternatively, the germination treatment can be applied and can comprise allowing the su1 cereal kernels to contact with water and/or to be placed in a high-humidity environment prior to processing.

The germination treatment can comprise various types of germination treatment and/or degrees of germination treatment. The type(s) and/or degree(s) of germination treatment can be selected based on a desired qualities of the end product (e.g., wall material, emulsifier, or composition).

In certain embodiments, the germination treatment comprises soaking the su1 kernels in water and/or any other aqueous system. The kernels can be soaked at a specific temperature (e.g., a temperature selected to achieve a certain degree of germination within a defined period of time) and/or for a specific time duration. In certain embodiments, the germination treatment comprises a moisture treatment of the su1 kernels at a set humidity level and/or temperature for a specific time duration. The pH and/or ionic strength of the water and/or other aqueous system employed for germination treatment can also be modified as desired to control and/or affect the germination process. In certain embodiments, the pH of the aqueous system can be between about 2 and about 11. In certain embodiments, the pH of the aqueous system is at or near a neutral pH (i.e., at or near a pH of 7). Additionally or alternatively, additives (e.g., salt(s) and/or hormone(s)) can be included in the aqueous system to affect the germination process. In certain embodiments, the germination treatment comprises any combination of the aforementioned germination variables, with the parameters for each controlled variable established as desired to achieve a specific quality of the end product and/or impart a characteristic in the end product (e.g., to optimize germination degree to increase emulsification properties of the wall material or emulsifier, or to select the type and/or amount of soluble proteins in the wall material or emulsifier).

After the germination treatment, the wet kernels (or its various developmental outcomes such as seedlings or sprouts) can be directly processed using the desired preparation approach to prepare the wall material or emulsifier, or dried before processing to prepare the wall material or emulsifier.

Various combinations of the germination treatment and processing approaches can be employed. In certain embodiments, a portion or an isolate of a su1 cereal is obtained by a method comprising no germination treatment and a dry processing approach. For example, whole su1 cereal grains (or kernels) without germination treatment can be processed into grits or flours via dry processing. Alternatively, specific kernel fractions (e.g., endosperm-enriched fractions) from kernels without germination treatment can be processed into grits or flours via dry processing.

In certain embodiments, a portion or an isolate of a su1 cereal is obtained by a method comprising no germination treatment and a wet processing and/or extraction approach. Whole su1 cereal grains without germination treatment can be processed via a wet extraction technique and thereafter, for example, spray-dried or vacuum-dried into a solid. Alternatively, specific kernel fractions (e.g., endosperm-enriched fractions) from kernels without germination treatment can be processed via a wet extraction technique and, for example, spray-dried or vacuum-dried into a powder.

In certain embodiments, a portion or an isolate of a su1 cereal is obtained by a method comprising germination treatment and a dry processing approach. For example, whole su1 cereal grains can be subjected to germination treatment as described herein, and thereafter dried and processed into grits or flours via dry processing. Alternatively, specific kernel fractions (e.g., endosperm-enriched fractions) from kernels subjected to germination treatment can be dried and processed via dry processing into grits or flours.

Still further, a portion or an isolate of a su1 cereal can be obtained by a method comprising germination treatment and wet processing and/or extraction approach. For example, whole su1 cereal grains can be subjected to germination treatment as described herein, and thereafter processed via wet extraction techniques. Thereafter, the extracted material can be spray-dried or vacuum-dried, for example, to obtain the wall material or emulsifier. Alternatively, specific kernel fractions (e.g., endosperm-enriched fractions) from kernels subjected to germination treatment can be processed via wet processing and/or extraction techniques. The product of the wet extraction can then be additionally processed as is known in the art into, for example, a drum-dried powder or the like.

### Properties of Wall Material or Emulsifier

Referring back to the wall materials or emulsifiers, the portion or isolate from su1 cereal can comprise PG, soluble proteins, and optionally, one or more saccharides. In certain embodiments, the portion or isolate is prepared from kernels of su1 corn using a water-based extraction technique (e.g., such techniques that are known in the art), followed by spray-drying to collect a solid end product. In certain embodiments, the resulting portion or isolate, in the form of a powder, can contain (by weight) about 10-99.9% PG, about 0-60% sugars (e.g., saccharides), and about 0.1-10% soluble proteins. In certain embodiments, the resulting portion or isolate contains (by weight) about 60% PG, about 20% sugars, and about 2% soluble proteins. As used herein, “soluble” components (e.g., soluble proteins) means those that are not precipitated after centrifugation at 11,000×g for 15 minutes.

In certain embodiments, the portion or isolate from su1 cereal comprises (by weight) greater than about 10% PG (such as, greater than 10% PG). For example, the portion or isolate can comprise at least about 10% PG, 20% PG, 30% PG, 40% PG, 50% PG, 60% PG, 70%, 80% PG, 90% PG, 99.9% PG, or higher, by weight.

In certain embodiments, the portion or isolate from su1 cereal comprises greater than about 0.1% soluble protein (such as, greater than 0.1% soluble protein) by weight. In certain embodiments, the portion or isolate comprises greater than about 0.5% soluble protein by weight.

In certain embodiments, the portion or isolate from su1 cereal comprises less than about 60% saccharides (such as, less than 60% saccharides) by weight. For example, the portion or isolate can comprise (by weight) less than 60%, less than 50%, less than 40%, less than 30%, less than 20%, less than 10%, or less than 1% saccharides. The saccharides can have a degree of polymerization (DP) equal to or lower than 2. The saccharides can comprise sucrose or glucose.

In certain embodiments, the portion or isolate does not comprise a detectable amount of saccharides (e.g., where the sugars or saccharides are removed through an ultrafiltration or other process known in the art).

In certain embodiments, the portion or isolate from su1 comprises greater than about 10% PG, greater than about 0.1% soluble proteins, and less than about 60% saccharides. In certain embodiments, the portion or isolate comprises greater than about 20% PG, greater than about 0.5% soluble proteins, and less than about 50% saccharides. In certain embodiments, the portion or isolate comprises greater than about 40% PG, greater than about 0.5% soluble proteins, and less than about 30% saccharides.

The portion or isolate from su1 cereal can further comprise about 0-80% of starch. In certain embodiments, the portion or isolate does not comprise starch. In certain embodiments, the portion or isolate comprises 5%, 10%, 15%, 20%, 25%, 30%, 35%, 40%, 45%, 50%, 55%, 60%, 65%, 70%, 75%, or 80% starch by weight. In certain embodiments, the portion or isolate comprises less than about 40% starch.

The portion or isolate from su1 cereal can be used as a wall material or emulsifier to encapsulate one or more core materials. In the flavor industry, for example, the core material can be flavor or fragrance ingredients that are encapsulated within a wall material and the product formed can serve to retain the aroma in foods during storage, protect the flavor from undesirable interactions, minimize flavor/flavor interactions, guard against either light-induced reactions or oxidation, and/or in certain instances, provide a controlled release of flavor.

The core material can be, for example, one or more of a food ingredient, a pharmaceutical compound, an oil, a nutrient (e.g., a vitamin), a fine chemical, an agricultural or crop care chemical, a preservative, a colorant, a flavor, an enzyme, a microorganism (e.g., probiotic bacteria), and/or an active ingredient. The core material can be one or more lipophilic components, one or more hydrophilic components, or one or more macro-, micro-, or nanoparticles not soluble or dispersible in either water or oil.

In certain embodiments, the portion or isolate from su1 cereal can be (e.g., have the properties of) an emulsifier (e.g., can form emulsions with oils or lipids). In certain embodiments, the portion or isolate from su1 cereal can be mixed with a lipid- or oil-based core material to form an emulsion. Accordingly, emulsions are provided that comprise the portion or isolate from su1 cereal and one or more core materials. In certain embodiments, the emulsion can further comprise a surfactant.

Most conventional wall materials do not exhibit the properties of an emulsifier and, as such, where a core material is oil- or lipid-based, an emulsion must first be formed through the addition of an emulsifier prior to encapsulation using the wall material. However, as the portion or isolate from su1 cereal is itself an emulsifier, where a wall material comprising a portion or isolate from su1 cereal is used to encapsulate a core material that is oil- or lipid-based, a distinct step of adding additional emulsifiers is not required (although they can be added if desired).

### Compositions

Compositions are provided that comprise the portion or isolate from su1 cereal as a wall material or emulsifier. As used herein, the term “composition” generally refers to a material system that comprises a wall material or emulsifier and a core material, with or without any other components. The composition can be in any form such as a solid, semi-solid, gel, syrup, paste, granulates/small beads, syrup, liquid, foam, or a combination thereof.

In certain embodiments, the composition comprises the portion or isolate from su1 cereal as the wall material or emulsifier and one or more core materials. For example, the core material can be one or more food or beverage components, dietary supplement components, feed components, cosmetic components, personal care components, home care components, agricultural or crop care components, and/or pharmaceutical components. The core material can also be one or more nutrients, one or more colorants, one or more flavors, one or more oils, one or more enzymes, one or more microorganisms, one or more active ingredients, or one or more fine chemicals. In certain embodiments, the one or more core materials comprises a combination of several different ingredients.

In certain embodiments, the composition is encapsulated via a spray-drying process. In certain embodiments, the composition is encapsulated via a freeze-drying process. In certain embodiments, the composition is encapsulated via a drum-drying process. In certain embodiments, the composition is encapsulated via a vacuum-drying process. Such processes and their protocols are well known in the relevant art and it will be appreciated that the composition can be produced using any encapsulation process now known or hereinafter developed.

In certain embodiments, the composition is in solid form. In certain embodiments, the composition is in semi-solid form. In certain embodiments, the composition is in gel form. In certain embodiments, the composition is in syrup or paste form. In certain embodiments, the composition is in liquid form. In certain embodiments, the composition is in foam form.

the composition can be concentrated. For example, it may be desirable to concentrate the composition prior to drying or the like (e.g., freeze drying or spray drying). Concentrating the composition can be performed using known techniques and/or equipment known in the art, for example, such as using a commercially available concentrator. In certain embodiments, the composition can be diluted using techniques and/or equipment known in the art. In certain embodiments, the composition is used in formulations of food, feed, dietary supplement, cosmetic, personal care, home care, pharmaceutical, agricultural or crop care, construction and painting, and/or other products.

Methods of Preparing the Portion or Isolate from Su1 Cereal and Compositions Thereof

The portion or isolate from su1 cereal can be water-soluble or water-dispersible solids prepared from an endosperm-containing fraction. In certain embodiments of preparing a (micro)encapsulated product (i.e., a composition), the portion or isolate is hydrated (e.g., dispersed in an aqueous solvent such as water) and mixed with one or more core materials. Thereafter, the mixture can be further processed to produce a desired end product. In certain embodiments, the further processing comprises encapsulation techniques.

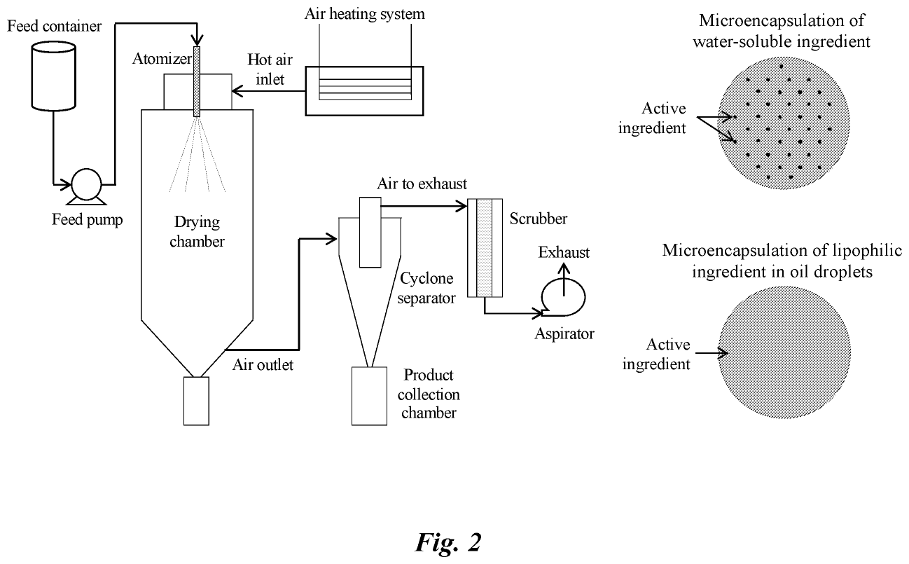

There are several principal techniques for encapsulation, including spray-drying, spray-chilling, extrusion, fluidized-bed coating, and coacervation that can be used to produce the compositions. Spray-drying (FIG. 2) is one of the most common and cost-effective encapsulation techniques. Equipment is readily available with very low production costs; for example, the cost of spray-drying is 30-50 times cheaper than that of freeze-drying (see Gharsallaoui et al., Applications of spray-drying in microencapsulation of food ingredients: An overview, Food Res Int'l 40: 1107-1121 (2007)).

In a typical spray-drying operation, a liquid is atomized in a current to lose solvent instantaneously and form a powder. There are typically three fundamental steps involved in spray-drying: 1) atomization of a liquid feed into fine droplets; 2) mixing these spray droplets with a heated gas stream to allow the liquid to evaporate and leave dried solids; and 3) separating and collecting the resulting dried powder from the gas stream.

The liquid feed can be a solution, dispersion, suspension, or emulsion. Atomization of the liquid feed forms small droplets to maximize heat and mass transfers. The size of particles typically increases with increasing viscosity.

Following atomization, the contact between droplets and the gas initiates drying. The gas used is typically air when water is the liquid solvent, or where the solvent is flammable, an inert gas such as nitrogen can be used. Spray-drying can involve the use of large quantities of air, usually at an elevated temperature of, for example, about 120-230° C. to provide a solid coating or matrix surrounding the core material. Furthermore, the air inlet temperature can be as high as 180° C. or even higher, and the outlet temperature can, in certain instances, be as low as possible to minimize thermal degradation. The liquid in the droplets evaporates due to the heat transfer from gas to droplets and liquid diffusion from droplet core to surface. With the reduction of moisture, a dry crust is formed at the droplet surface, which leads to a rapidly reduced rate of drying.

After the drying stage, the dried particles can be separated from the airflow in a cyclone, and ultra-fine particles are retained in a device such as a baghouse. Associated with the composition and liquid content of the droplets, the spray-dried particles can have different porosities. (see Gharsallaoui (2007), supra).

In certain instances, spray-dried particles can be as small as several microns in size, thus forming a fine powder.

In certain embodiments, an endosperm-enriched fraction can be used to form a composition using a method for encapsulating one or more core materials. Such a method can comprise preparing a liquid (e.g., a solution, dispersion, suspension, or emulsion) of one or more core materials; hydrating and/or dispersing an endosperm-enriched fraction with/in the prepared liquid to form a mixture; and processing the mixture to generate (micro)encapsulation solids. In certain embodiments, the processing step can comprise spray-drying, freeze-drying, or drum-drying the mixture pursuant to protocols known in the art.

The method can optionally include a homogenization step. For example, in certain embodiments, the method can comprise homogenizing the mixture prior to the processing step. Additionally or alternatively, the method can optionally include removing particles larger than a specific diameter from the mixture prior to the processing step.

The endosperm-enriched fraction can be any type of kernel particles or fraction that comprises an endosperm component (e.g., PG) in an amount greater than the amount of such component in the whole kernel. In certain embodiments, the endosperm-enriched fraction is obtained from a su1 cereal kernel using any methods now known or hereinafter developed in the relevant art. For example, a degerming process of kernels of su1 cereal can be used to prepare endosperm-enriched fractions.

In certain embodiments, a spray-drying method for encapsulating one or more core materials is provided. The method can comprise providing (or forming) a liquid feed comprising a portion or an isolate from su1 cereal and at least one core material; homogenizing the liquid feed; and spray-drying the homogenized liquid feed to form a particular product (e.g., wherein the particular product comprises the core material encapsulated by the wall material or emulsifier). In some embodiments, the liquid feed is an aqueous emulsion and at least one of the core materials is lipophilic. Additionally or alternatively, at least one of the core materials can be hydrophilic. The core material can be any of the core materials described herein.

The solution of the liquid feed can be formulated to have a particular viscosity. In certain embodiments, the solution comprises a 30% dispersion of the wall material or emulsifier in the solvent (e.g., water), and has a viscosity of not greater than about 500 mPas (such as 500 mPas). In certain embodiments, the solution comprises a 30% dispersion of the wall material or emulsifier in the solvent (e.g., water), and has a viscosity of not greater than about 200 mPas (such as 200 mPas). In certain embodiments, the solution comprises a 30% dispersion of the wall material or emulsifier in the solvent (e.g., water), and has a viscosity of not greater than about 100 mPas (such as 100 mPas), or not greater than about 150 mPas (such as 150 mPas).

The particular product can be a powder. The particular product can be a spray-dry (micro)encapsulation (SD-M) solid.

The wall material or emulsifier can be any of the wall materials or emulsifiers described herein. In certain embodiments, the wall material or emulsifier is compatible with the core material, provides a desired rate of dissolution and core release, and/or presents a suitable desired size.

Methods for preparing an emulsion are also provided. A method for preparing an emulsion can comprise providing a mixture of at least one core material dispersed in a solution, the solution comprising a solvent and a wall material or emulsifier, wherein the wall material or emulsifier comprises: a portion or an isolate from su1 cereal; and greater than about 10% phytoglycogen, greater than about 0.1% soluble proteins, and less than about 60% saccharides with a DP equal to or lower than 2. In certain embodiments, at least one of the core materials is lipophilic.

The cereal can be corn, rice, barley, or sorghum, and it can be subjected to a germination treatment. In some instances, the cereal is not subjected to a germination treatment. The su1 cereal can comprise endosperm fraction. The portion or isolate can comprise any of the embodiments described herein including, for example, a portion or isolate comprising greater than about 20% phytoglycogen, greater than about 0.5% soluble proteins, and less than about 50% saccharides with a DP equal to or lower than 2. In certain embodiments, the portion or isolate further comprises less than about 80% starch, and optionally less than about 40% of starch. In certain embodiments, the portion or isolate comprises about 0-80% of starch.

It will be appreciated that the methods described herein can be used to produce food products, nutraceuticals, personal care products, and/or dietary products, for example. In certain embodiments, a food product, a nutraceutical, or a dietary product can be produced using the methods hereof. In certain embodiments, a food product, a nutraceutical, or a dietary product is provided comprising a portion or an isolate of su1 cereal or a composition hereof. A cosmetic, a personal care, or a home care product can also be provided that comprises a portion or an isolate of su1 cereal or a composition hereof (and/or produced using the methods hereof). A pharmaceutical product can be provided that comprises a portion or an isolate of su1 cereal or a composition hereof (and/or produced using the methods hereof). A crop care or agricultural product can be provided that comprises a portion or an isolate of su1 cereal or a composition hereof (and/or produced using the methods hereof).

Those skilled in the art will recognize that numerous modifications can be made to the specific implementations described herein. The implementations should not be limited to the particular limitations described as other implementations can be possible.

While this disclosure has been illustrated and described in detail in the drawings and description, the same is to be considered as illustrative and not restrictive in character, it being understood that only certain embodiments have been shown and described and that all changes and modifications that come within the spirit of the disclosure are desired to be protected.

It is intended that the scope of the present methods and compositions be defined by the following claims. However, it must be understood that this disclosure can be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### Certain Additional Definitions

As used herein, the following terms and phrases shall have the meanings set forth below.

Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

All publications, patents, and patent documents referred to in this document are incorporated by reference herein in their entirety, as though individually incorporated by reference.

In the event of inconsistent usages between this document and those documents so incorporated by reference, the usage in the incorporated reference should be considered supplementary to that of this document; for irreconcilable inconsistencies, the usage in this document controls.

### EXAMPLES

The following examples serve to illustrate the present disclosure. The examples are not intended to limit the scope of the claimed invention.

### Example 1

### Production of Su1 Extract

Partially degermed kernel particles of Silver Queen, a sweet corn variety of su1 genotype, were prepared. The amount of corn particles was about 121 kg. The corn particles were mixed with 363 kg of water, and the mixture was stirred for about 2.5 hours at ambient temperature. The mixture was then processed to remove insoluble particles, including brans, germs, and starch, using commercially available equipment pursuant to protocols known in the art. The liquid collected was spray dried at an inlet temperature of 175-180° C. and outlet temperature of 75-80° C. to collect su1 extract powder.

While a portion of material was lost in the processing, about 32 kg of spray-dried su1 extract powder was collected. The moisture content of collected powder was about 5.56%. For vegetable oil, the su1 extract powder had a better emulsification property than acacia gum. The powder was stored in a 4° C. cooler.

### Example 2

### Germination of Su1 Kernels (Seeds)

In general, germination leads to activated proteases, increased protein solubility and digestibility, and altered carbohydrate structure and profiles (see Omary et al., Effects of germination on the nutritional profile of gluten-free cereals and pseudocereals: a review, Cereal Chem 89: 1-14 (2012); Yi et al., Germination of sorghum grain results in significant changes in paste and texture properties, J Texture Studies 48: 386-391 (2017); and Horstmann et al., A comparative study of gluten-free sprouts in the gluten-free bread-making process, European Food Res & Tech 617-62: 245 (2019)). Considering the potential impact of germination on protein profile of su1 kernels and thus on the properties of su1 extracts, a germination test was conducted.

For the germination treatment, su1 corn kernels (seeds) were first soaked in water for 24 hours at room temperature. Thereafter, the excess water was decanted and imbibed kernels were placed on 10-mesh stainless steel sieves, covered with wet paper towels to maintain a high moisture content, and aerated at room temperature for up to 72 hours. After either 24 hours or 72 hours of aeration, kernels were collected and crushed in an extraction water. The mixtures were thereafter passed through a sieve to remove larger particles and the permeates (i.e., fluid passing through the sieve) were collected (which contained phytoglycogen (PG), soluble proteins, and sugars), pooled, and centrifuged at 11,000×g for 15 minutes. The supernatant was collected and spray-dried using Mini Spray Dryer B-290 (Buchi Labortechnik AG, Easter Switzerland, Switzerland) to produce su1 extracts in powder form.

The su1 corn kernels that were not subjected to germination treatment were also subjected to water extraction, particle removal, and spray-drying to collect powder in accordance with the above-described protocol.

The su1 extracts obtained were identified as follows:
(1) extract-1, the su1 corn extract from kernels not subjected to germination treatment;
(2) extract-2, the su1 corn extract from kernels subjected to the germination treatment comprising 24 hours soaking and 24 hours aeration in high humidity; and
(3) extract-3, the su1 corn extract from kernels subjected to the germination treatment comprising 24 hours soaking and 72 hours aeration in high humidity.

The content of soluble proteins of each su1 corn extract was determined via a Bradford assay with the results are shown in Table 1.

TABLE 1
Content of Soluble Proteins and Number of Proteins
Detected in Proteomic Analysis for su1 Extracts

| | Content of soluble | Number of proteins detected |
|---|---|---|
| Test Group | proteins | in proteomic analysis |
| Extract-1 | 1.96% | 105 |
| Extract-2 | 1.26% | 89 |
| Extract-3 | 0.82% | 13 |

### Example 3

### Proteomic Analysis

Extract-1, extract-2, and extract-3 were each subjected to proteomic analysis using gel separations and mass spectrometry. The number of proteins detected are shown in Table 1.

### Example 4

### Viscosity of Su1 Extracts

The viscosity of a (water) dispersion that contained 30% su1 corn extract (by weight) was tested using a viscometer and the triplicate readings were 53, 53, and 53 mPas. In contrast, readings were 303, 309, and 301 mPas for 30% acacia gum and 28, 27, and 28 for 30% Hi-Cap 100. Low viscosity is essential for su1 extract to be used as a wall material.

### Example 5

### Microencapsulation of Water-Soluble Ingredients

su1 corn extract was used to encapsulate the components in anthocyanins-containing blueberry fluid or betalains-containing beet juice. Both anthocyanins and betalains are important nutrients and/or food colors and both are highly susceptible to degradations.

Primarily, blueberries were assessed as an example of food materials that contain water-soluble active ingredients. As noted above, blueberry has high content of anthocyanins. Anthocyanins are a group of water-soluble pigments belonging to the flavonoid group and contribute to the red, purple, and blue coloring of plants, and they are susceptible to degradation and changes affected by pH, temperature, light, oxygen level, metal ions, and presence of other compounds (see Khoo et al., Anthocyanidins and anthocyanins: colored pigments as food, pharmaceutical ingredients, and the potential health benefits, Food & Nutrition Res 61: 1361779 (2017); and Mukherjee, Phyto-Pharmaceuticals, nutraceuticals and their evaluation, in quality control and evaluation of herbal drugs, edited by Mukherjee P K, Elsevier (2019)).

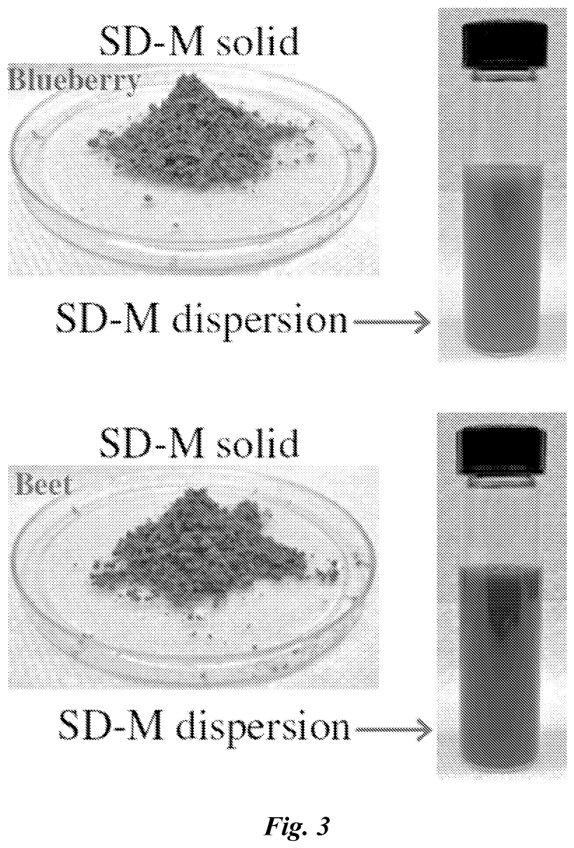

The blueberry fluid was generated from frozen whole blueberry fruits using a proprietary approach. Degermed Silver Queen su1 corn kernel particles (grits and flour) were then added to the blueberry fluid. After soaking, the mixture was blended using a high-speed blender (i.e. a food processor) and the mixture was passed through a sieve to remove blueberry seeds and other large particles. The resulting filtrate was spray-dried to collect the powder (FIG. 3), and the powder was rehydrated to obtain a dispersion (FIG. 3).

Beets were also assessed as an example for food materials that contain water-soluble active ingredients. Betalains found in beets are a class of water-soluble natural pigments and contain two categories, betacyanins and betaxanthins that are often used as food coloring to give a reddish color (see Coy-Barrera et al., Analysis of betalains (betacyanins and betaxanthins), in Recent Advances in Natural Products Analysis, edited by Silva et al., Elsevier (2020)).

Fresh beetroots were cleaned, cut into cubes, and subjected to high-speed blending using a food processor. The puree obtained was washed on a sieve using water. To the filtrate collected, degermed Silver Queen su1 corn kernel particles (grits or flour) were added. After soaking, the mixture was subjected to high-speed blending and passed through a sieve. The resulting filtrate was spray-dried to collect the powder (FIG. 3), and the powder was rehydrated to obtain a dispersion (FIG. 3).

### Example 6

### Emulsification and Encapsulation of Lipophilic Ingredients

su1 corn extracts were used to emulsify and spray-dry paprika oleoresin oils. Paprika oleoresin is an oil-soluble extract from Capsicum annuum and commonly used as food colorings.

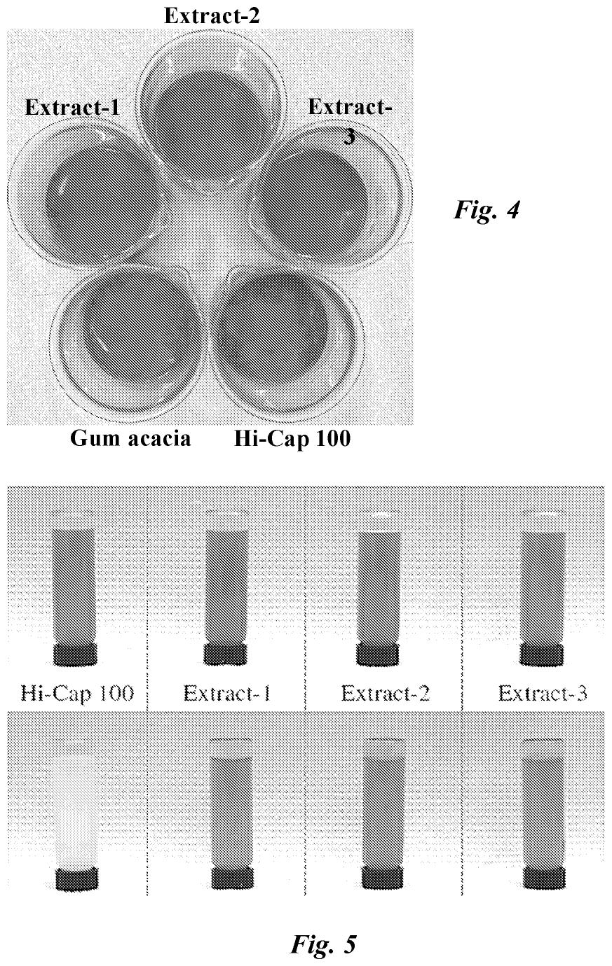

Emulsions were prepared using paprika oleoresin and individual wall materials or emulsifiers, including su1 corn extract-1, extract-2, and extract-3, Hi-Cap-100 (a commercial OSA-starch), and acacia gum. It was found that: (1) paprika oleoresin is a highly viscous oil that formed scum in all crude emulsions (after blending using a food processor), and (2) after homogenization using a 2-stage, high-pressure homogenizer, emulsions formed by Hi-Cap 100 and acacia gum still showed a high amount of scum at surface, whereas su1 corn extract-1 showed a much lower amount of scum, and su1 corn extract-2 and extract-3 did not show any scum (FIG. 4).

The scum at emulsion surface indicates a less than sufficient emulsification capability which may cause operational challenges due to the attachment of scum to the flow path of homogenizer and spray-dryer. Therefore, for the emulsification and spray-dry (micro)encapsulation operations of paprika oleoresin, su1 corn extract-2 and extract-3 were identified as the best performers, extract-1 was acceptable, and Hi-Cap 100 and acacia gum were problematic.

FIG. 5 shows the chemical stability of emulsions diluted from the paprika oleoresin emulsions shown in FIG. 4. The initial color strength in all emulsions was the same (top row of FIG. 5). After being placed under a bench light (about 310 lux) for 5 days, the emulsion formed by Hi-Cap 100 showed a complete loss of red color, whereas the emulsions formed by su1 corn extract-1, extract-2, and extract-3 showed reduced but considerable color strength (bottom row of FIG. 5). That the su1 extracts showed such a superior capability in protecting the chemical nature of paprika color in emulsions was noteworthy and surprising.

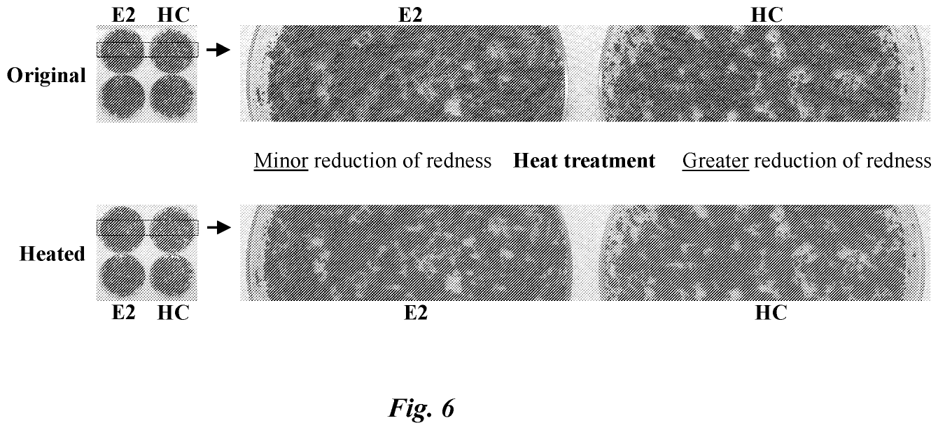

In addition to the forgoing, the paprika oleoresin emulsions prepared using su1 corn extract-2 and Hi-Cap 100 as emulsifiers and wall materials were spray-dried, each with wall-to-oil ratio of 2:1, to collect spray-dried solids. The collected solids were heated at 70° C. or 8 hours and then allowed to cool to room temperature in 16 hours. FIG. 6 shows the color of spray-dried solids of paprika oleoresin with each of extract-2 or Hi-Cap 100 as wall material. A comparison among the color of solids showed that, after heat treatment, the reduction of red color intensity was greater for Hi-Cap 100 than for extract-2, suggesting a greater capability of extract-2 than Hi-Cap 100 to protect active compounds in the spray-dried solids.

### Example 7

### Emulsification and Encapsulation of Lipophilic Ingredients

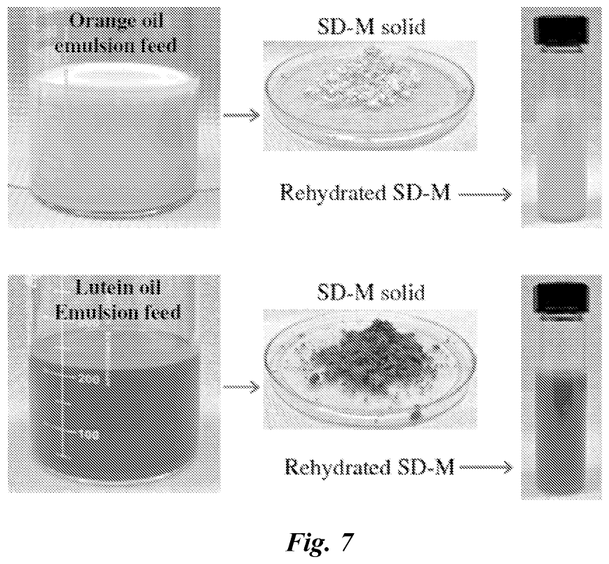

Orange oil is an essential oil extracted from oil sacs in the rind of oranges. Lutein is a xanthophyll and natural carotenoid that exhibits a yellowish to orange color. As shown in FIG. 7, emulsions of orange and lutein oils were prepared using a su1 corn extract. In the orange oil emulsion, the oil and su1 extract content was both 14.3%. In the lutein oil emulsion, the oil and su1 extract content was 7% and 19.6%, respectively. Emulsions were spray-dried to form powders using the protocols set forth herein, and the powders were rehydrated to form even and smooth emulsions.

1. A wall material or emulsifier comprising a portion or an isolate from sugary-1 cereal, wherein the portion or isolate comprises greater than about 10% phytoglycogen, greater than about 0.1% soluble proteins, and less than about 60% saccharides with a degree of polymerization (DP) equal to or lower than 2.

2. The wall material or emulsifier of claim 1, wherein said cereal is corn, rice, barley, or sorghum.

3. The wall material or emulsifier of claim 1, wherein said portion or isolate comprises greater than about 20% phytoglycogen, greater than about 0.5% soluble proteins, and less than about 50% saccharides with a DP equal to or lower than 2.

4. (canceled)

5. The wall material or emulsifier of claim 1, wherein said portion or isolate further comprises about 0-80% of starch.

6. The wall material or emulsifier of claim 1, wherein said portion or isolate further comprises less than about 40% of starch.

7. The wall material or emulsifier of claim 1, wherein said sugary-1 cereal is not subjected to germination treatment.

8. The wall material or emulsifier of claim 1, wherein said sugary-1 cereal is subjected to a germination treatment.

9. The wall material or emulsifier of claim 1, wherein a portion or an isolate from sugary-1 cereal comprises endosperm fractions.

10. (canceled)

11. The wall material or emulsifier of claim 1, encapsulating one or more core materials comprising one or more lipophilic components or one or more hydrophilic components, or emulsifying one or more oils, fats, or lipophilic materials.

12. (canceled)

13. The wall material or emulsifier of claim 1,
encapsulating one or more core materials, wherein the core materials are one or more of an ingredient in food or beverage, an ingredient in dietary supplement, an ingredient in feed or animal food, an ingredient in cosmetics, personal care, home care, health care, or crop care product, a pharmaceutical compound, an oil, fat, lipophilic material, a nutrient, a fine chemical, an ingredient in agricultural applications or crop care, a preservative, a colorant, an enzyme, a microorganism, a flavor, and/or an active ingredient; or
emulsifying oils, fats, or lipophilic materials, wherein the oils, fats, or lipophilic materials are one or more of an ingredient in food or beverage, an ingredient in dietary supplement, an ingredient in feed or animal food, an ingredient in cosmetics, personal care, home care, health care, or crop care product, a pharmaceutical compound, a nutrient, a fine chemical, an ingredient in agricultural applications or crop care, a preservative, a colorant, an enzyme, a microorganism, a flavor, and/or an active ingredient.

14-19. (canceled)

20. The wall material or emulsifier of claim 1, wherein a 30% dispersion of said wall material or emulsifier in water has a viscosity not greater than about 500 mPas.

21. A composition comprising:
a wall material or emulsifier separated or isolated from sugary-1 cereal; and
one or more core materials, oils, fats, or lipophilic materials mixed with, emulsified by, stabilized by, coated by, entrapped within, encapsulated by, and/or encapsulated and stabilized by the wall material or emulsifier;
wherein said wall material or emulsifier comprises greater than about 10% phytoglycogen, greater than about 0.1% soluble proteins, and less than about 60% saccharides with a degree of polymerization (DP) equal to or lower than 2.

22. The composition of claim 21, wherein the wall material or emulsifier comprises greater than about 20% phytoglycogen, greater than about 0.5% soluble proteins, and less than about 50% saccharides.

23. The composition of claim 21, wherein the one or more core materials, oils, fats, or lipophilic materials comprises one or more food or beverage, dietary supplement, feed or animal food, cosmetic, personal care, home care, crop care or agricultural, or pharmaceutical components, one or more nutrients, one or more colorants, one or more flavors, one or more oils, fats, or lipophilic materials, one or more enzymes, one or more microorganisms, one or more active ingredients, one or more fine chemicals, or a combination of any of the foregoing.

24-27. (canceled)

28. The composition of claim 21, wherein the composition is spray-dried.

29. A method of encapsulating one or more core materials comprising:
providing a liquid feed comprising one or more core materials dispersed in a solution, the solution comprising a solvent and a wall material or emulsifier, wherein the wall material or emulsifier comprises:
a portion or an isolate from sugary-1 cereal; and
greater than about 10% phytoglycogen, greater than about 0.1% soluble proteins, and less than about 60% saccharides with a degree of polymerization (DP) equal to or lower than 2; and
spray-drying the liquid feed to form a solid product.

30. The method of claim 29, wherein the liquid feed is an aqueous emulsion and at least one of one or more core materials is lipophilic.

31. The method of claim 29, wherein at least one of one or more core materials is hydrophilic.

32. The method of claim 29, wherein the portion or isolate from sugary-1 cereal is from a cereal subjected to a germination treatment.

33. (canceled)

34. The method of claim 29, wherein a portion or an isolate from sugary-1 cereal comprises endosperm fractions.

35. (canceled)

36. The method of claim 29, wherein the solid product is in a powder or granulated form.

37. The method of claim 29, wherein the solvent of the liquid feed comprises water.

38-41. (canceled)

42. The composition of claim 21, wherein the composition is a liquid, a syrup, a paste, a gel, a solid, a powder, or an inhalant.

43-51. (canceled)
